(19) United States
(12) Patent Application Publication
(10) Pub. No.: US 2026/0306725 A1
(43) Pub. Date: Oct. 8, 2026

(54) DEVICE FOR PRODUCING DIALYSIS LIQUID

(71) Applicant: B. Braun Avitum AG, Melsungen (DE)

(72) Inventors: Guenter Niemetz, Melsungen (DE); Ralf Kuchenbaecker, Baunatal (DE)

(21) Appl. No.: 18/874,639

(22) PCT Filed: Jun. 14, 2023
(86) PCT No.: PCT/EP2023/065915

§ 371 Date: Dec. 13, 2024

(30) Foreign Application Priority Data
Jun. 14, 2022 (DE) 10 2022 114 956.0

(51) Int. Cl. A61M 1/16

(52) U.S. Cl. CPC A61M 1/1656; A61M 2205/10; A61M 2205/3317; A61M 2205/3365; A61M 2205/50

(57) ABSTRACT

A device for or of an extracorporeal blood treatment machine for producing and/or conditioning a dialysis liquid includes a fluid-carrying main line for introducing osmotic water, and at least one fluid-carrying secondary line attached to the fluid-carrying main line for feeding an additive into the osmotic water. At least one metering pump, in an intake phase/intake stroke, sucks the additive out of at least one storage container and, in a serially subsequent discharge phase/discharge stroke, expels the additive into the osmotic water. A stepper motor operates the metering pump, and an open-loop or closed-loop control unit controls or regulates the stepper motor. The open-loop or closed-loop control unit controls or regulates the stepper motor in such a way that the intake phase is shorter than the discharge phase.

### CROSS-REFERENCE TO RELATED APPLICATION(S)

This application is the United States national phase entry of International Application No. PCT/EP2023/065915, filed on Jun. 14, 2023, and claims priority to German Application No. 10 2022 114 956.0, filed on Jun. 14, 2022. The contents of International Application No. PCT/EP2023/065915 and German Application No. 10 2022 114 956.0 are incorporated by reference herein in their entireties.

### FIELD

The present disclosure relates to a device for an or of an extracorporeal blood treatment machine for producing and/or processing a dialysis liquid while (simultaneously) reducing a conductivity fluctuation in the ion-mixed osmotic liquid (osmosis water). Furthermore, the present disclosure relates to a driving/regulating method of a metering pump for producing and/or processing dialysis liquid.

In other words, the present disclosure relates to a device and method for producing dialysis liquid in which acidic and/or basic components are added to an osmosis water in as precise a manner as possible.

### BACKGROUND

In extracorporeal blood treatment, for example hemodialysis, hemofiltration, hemodiafiltration, etc., the dialysis liquid is produced from the fundamental components of osmotic water, basic fluid and acidic fluid or acetate via a dialysis machine. During the dialysis treatment of a patient, the dialysis liquid is passed through the dialysis fluid-side chamber of a dialyzer and uremic toxins and water are absorbed by diffusion (hemodialysis) or diffusion in combination with convection (hemofiltration or hemodiafiltration) via its semipermeable membrane from the blood that is passed through the blood-side chamber of the dialyzer.

During extracorporeal blood treatment, the patient's blood to be treated is passed through semi-permeable hollow fiber membranes in the dialyzer, which are surrounded by the dialysis liquid. A basic (first) component of the dialysis liquid is usually a substrate with sodium hydrogen carbonate, an acidic (second) component is usually a solution with sodium chloride, potassium chloride, magnesium chloride, calcium chloride, glucose, acetic acid, and/or citric acid or only one component is used, which usually consists of acetate.

Metering pumps and conductivity probes are used to produce the dialysis liquid via a control loop. A probe measures the conductivity of the osmotic water/fundamental component of the dialysis liquid after the addition of the basic component via a metering pump and the actual value of the conductivity of the water mixed with the basic component is compared with a target value via a computing unit and, if necessary, is readjusted via the rotation speed of the metering pump. After the acidic component has been added via another metering pump, another probe measures the conductivity of the entire dialysis liquid, and the actual value of the conductivity of the entire dialysis liquid is compared with another target value via a computing unit and, if necessary, is readjusted via the rotation speed of this further metering pump.

In other words, the osmotic water is mixed with a basic and possibly an acidic component. Separate containers are provided for the basic and acidic components, which contain corresponding solutions with a specific concentration of substances. Metering pumps are furthermore provided, which pump the corresponding solutions out of the containers and add them to the osmotic water. It is very important that the mixing ratio is correct.

In order to ensure this, the electrical conductivity probes are used, which measure the electrical conductivity of the osmosis water before/after each addition of the individual solutions, wherein a computer can draw conclusions from the conductivity changes about the concentration of the substances currently being added, which are contained in the solutions. This means that, based on the conductivity measurement results, the computer is able to regulate the dosage of the individual solutions in which substances with a certain concentration are dissolved in such a way that the concentration of substances in the osmotic water assumes a certain value in each case.

Dosing regulations regulate both the disruptive influences, for example flow fluctuations of the osmosis water or air bubbles in the concentrate sources, as well as the conveying characteristics of the metering pumps over the entire controlled system. Since the metering pumps, such as rotary vane piston pumps or diaphragm pumps and the like, do not have continuous conveying characteristics, this leads to constant conductivity fluctuations that the control system can never fully settle. Instead, the metering pumps have an intake phase and a discharge phase. In the intake phase, a fluid is sucked in by the metering pump or a piston of the metering pump and in the discharge phase, the fluid is conveyed/discharged by the metering pump or the piston of the metering pump. This means that the metering pump cannot deliver during the intake phase. Therefore, the volume delivered by the metering pump has a sinusoidal curve with an intake stroke and a discharge stroke.

In order to reduce the disruptive influences caused by the discontinuous conveying characteristics of the metering pumps, additional mixing or steaming chambers are often installed, from which liquid can then be continuously withdrawn with an essentially constant mixture. However, these lead to additional costs and also increase the volume of liquid contained in the system.

The stepper motor control used consists of a driver that alternately energizes the phase windings of the stepper motor so that the stepper motor makes a rotational movement by one resolution step with each drive pulse. The resolution steps are determined by the mechanical construction of the windings of the stepper motor (e.g. 200 steps per revolution, i.e. 1.8° step angle per drive pulse).

DE 10 2018 010 174 A1 discloses a device for producing a dialysis liquid, wherein the device has a fluid line for supply a dialysis liquid and means which are configured to convey a dialysis liquid discontinuously in the fluid line, wherein the device has a conductivity sensor which is arranged to measure the conductivity of the dialysis liquid, wherein the device has a concentrate line which opens into the dialysis liquid line upstream of the conductivity sensor at a supply point and has a concentrate container from which the concentrate pump delivers concentrate into the concentrate line and from this into the dialysis-liquid line. Accordingly, a reduction of the conductivity fluctuations via a metering pump and subsequent conductivity probe is known.

DE 10 2010 015 293 A1 describes a hydraulic power steering system with a pump unit for generating volume flow pulsations and pressure pulsations. The pump unit is connected to a hydraulic fluid reservoir via an input line and to a steering valve via an output line. An adjustable flow valve is arranged in the output line and a control device is provided to control the flow valve.

DE 10 2014 109 369 A1 discloses a dialysis machine which has a processing system with a main line for dialysis liquid, at which several feed lines for respective concentrates open. A respective mixing chamber and then a respective conductivity probe are arranged downstream of each opening. A common pump is arranged downstream of the last conductivity probe. The negative pressure in the main line is set via a pressure control valve, which is located upstream of the first opening in the main line. Furthermore, a proportional valve or a digital switching valve is provided in each feed line for dosing the various concentrates. The pressure control valve and the proportional valves or switching valves are controlled electromagnetically by a central electronic control unit.

EP 3 843 806 A1 relates to a device and a method for producing a dialysis liquid for use in an extracorporeal blood treatment, wherein the device comprises a main line for supplying water, preferably osmosis water or ultrapure water, in the course of which an acid and a base liquid are each introduced in a specific dosage, wherein the dosage is adjusted by a control and regulation unit as a function of at least one chemical and/or physical parameter, preferably the conductivity, of the water/liquid mixture, and the at least one chemical and/or physical parameter is detected by a measuring device, in particular a conductivity measuring probe, wherein a first, preferably a single measuring device is arranged at a respective downstream portion of the main line of the introduction point for the acidic fluid and downstream of the introduction point for the basic fluid, and the control and regulation unit at least temporarily controls the introduction of the acidic fluid and the basic fluid in such a way that only one of the acidic and basic fluids is introduced into the main line over a predetermined period of time or a predetermined interval.

A metering pump is known from U.S. Pat. No. 6,457,944 B1, which is controlled in such a way that a discharge phase of the metering pump is longer than an intake phase. The metering pump is driven by an asynchronous motor to which a current with a lower frequency is applied during the discharge phase than during the intake phase. As a result, the rotational speed of the asynchronous motor is lower in the discharge phase than in the intake phase. This enables the metering pump to deliver fluid as evenly as possible.

In order to dispense the fluid through the metering pump as evenly as possible, the driving of the metering pump has to be synchronized with the suction and discharge phase of the metering pump. In order to do this, the position of the electric drive of the metering pump has to be detected. In the case of asynchronous motors as in U.S. Pat. No. 6,457,944 B1, the position of the motor is detected by a position encoder or rotary encoder. The start of the suction and/or discharge phase of the metering pump is additionally detected by sensors on the metering pump. The position of the asynchronous motor detected by the position encoder is then synchronized with the start of the suction and/or discharge phase of the metering pump in order to enable the most uniform fluid delivery possible. However, the use of a position encoder incurs costs and results in a higher control or regulation effort for the motor of the metering pump.

### SUMMARY

The object of the present disclosure is to optimize the preceding regulating or control circuit in such a way that a temporally stable electrical conductivity of the dialysis solution at or in the dialyzer is/can be generated with comparatively little regulation or control effort, thus ensuring a constant mixing ratio in the dialysis liquid flow.

This object is solved by a device for an or of an extracorporeal blood treatment machine for producing and/or processing a dialysis liquid, a driving or control method of a metering pump for producing and/or processing dialysis liquid, a computer-readable storage medium and a computer program.

Accordingly, the present disclosure relates to a device/regulation or control device for an or of an extracorporeal blood treatment machine for producing and/or processing a dialysis liquid. The device comprises a main fluid line for introducing osmotic water, at least one secondary fluid line connected to the main fluid line for supplying an additive into the osmotic water, and a metering pump arranged in the secondary fluid line, which sucks in the additive from a storage means in an intake phase/stroke and discharges the additive into the osmotic water within the main fluid line in a serially following discharge phase/discharge stroke.

Furthermore, the device has an electric drive/a drive unit for driving/operating the metering pump, and a control or regulation unit for controlling or regulating the electric drive, which controls or regulates the electric drive in such a way that the intake phase of the metering pump is shorter than the discharge phase of the metering pump. The electric drive is a stepper motor and the metering pump has a magnetic sensor via which a position signal of the metering pump, in particular a position signal of a piston of the metering pump, can be detected. The control or regulation unit synchronizes a start of the intake phase and/or of the discharge phase with the detected position signal.

In other words, the control or regulation unit may be configured and provided to control the stepper motor synchronously with a start of the intake phase and/or of the discharge phase by the detected position signal (detected by the magnetic sensor). In this context, synchronous driving can be understood to mean that the control or regulation unit changes the (drive) signal to the stepper motor, preferably the frequency of the current, when the intake phase and/or the discharge phase of the metering pump starts. That is, the control or regulation unit varies the current to the stepper motor when the metering pump has a phase change. This ensures effective smoothing of the fluid delivery of the metering pump. More specifically, the control or regulation unit may drive the stepper motor, i.e. apply a current to it, precisely when the metering pump starts or ends the intake phase and/or the discharge phase. The control or regulation unit may therefore use the detected position signal to recognize when the intake phase and/or the discharge phase starts (or ends) and control the stepper motor based on the position signal or the start of the intake phase and/or of the discharge phase.

The stepper motor as an electric drive may be characterized in particular by the fact that a (physical) position of the stepper motor is known, since the position of the stepper motor may be calculated from the number of steps taken. Stepper motors may also enable constant-speed or precisely adjustable operation without additional sensors and/or rotation-speed controllers.

The magnetic sensor may be attached to the metering pump in particular and detects the position signal of the metering pump.

In other words, the device produces dialysis liquid from the fundamental components (osmotic) water, basic fluid and possibly acidic fluid or only from acetate. For this purpose, at least the metering pump and at least one electrical conductivity probe are used via a regulating or control circuit. The metering pump feeds the at least one additive into the osmotic water in the main fluid line and is driven by the stepper motor for this purpose. The stepper motor as an electric drive is driven by the control or regulation unit or a driving unit of the control or regulation unit. The control or regulation unit controls/regulates the stepper motor of the metering pump in such a way that a desired concentration of the additive is set in the main fluid line downstream to the confluence point of the secondary fluid line. The concentration of the additive is preferably detected by the electrical conductivity probe via the electrical conductivity of the (mixed) fluid and is then preferably fed back to the control or regulation unit as feedback from the control loop. The metering pump has the intake phase and the discharge phase, which results in discontinuous (sinusoidal) conveying characteristics of the metering pump. The stepper motor is driven or regulated by the control or regulation unit in such a way that the duration from the start of the intake phase to the end of the intake phase and thus to the start of the discharge phase is shorter than the duration from the start of the discharge phase to the end of the discharge phase. More specifically, a duration of the intake stroke is shorter than a duration of the discharge stroke. However, the volume of fluid sucked in during the intake phase is equal to a volume of fluid delivered/discharged by the metering pump during the discharge phase.

Preferably, the metering pump is a piston pump or a diaphragm pump and therefore has one intake stroke and one discharge stroke.

The at least one metering pump may also be a first metering pump and a second metering pump, wherein the first metering pump conveys a first additive and the second metering pump conveys a second additive (independently of the first metering pump).

The stepper motor may be driven or regulated directly by the control or regulation unit. However, the stepper motor may also be driven by a drive unit, which may optionally be part of the control or regulation unit.

The essence of the disclosure is thus that a stepper motor of a metering pump is driven in such a way that an intake phase of the metering pump is shorter than a discharge phase of the metering pump in order to compensate for a fluctuating delivery volume of the metering pump. By using the stepper motor as the drive of the metering pump, the position of the stepper motor and thus of an actuator of the metering pump may be determined from the number of steps taken and the stepper motor may thus be driven synchronously with the start of the intake or discharge phase.

In this context, the (physical) position of the stepper motor may be understood in particular as a rotation of the stepper motor or a number of (completed) steps of the stepper motor. The position of the stepper motor may in particular be compared with a specified angle of rotation of an electric motor. In contrast to the angle of rotation of the electric motor, however, the position of the stepper motor may be detected without sensors by detecting the number of (completed) steps of the stepper motor.

By shortening the intake phase compared to the discharge phase, the duration of the intake stroke of the metering pump is shortened compared to the duration of the discharge stroke. This means that the metering pump discharges additive over a longer period of time than is sucked in by the metering pump. As a result, the additive is not released into the osmotic water all at once, but continuously over a certain period of time. By shortening the intake phase, the time during which no additive is added to the osmotic water is shortened. The conductivity probe, which detects the concentration of the additive in the osmotic water (in the main fluid line) after the metering pump, thus detects the addition of the additive over a longer period of time. This reduces the influence of the detected conductivity fluctuations on the controlling or regulating of the metering pump, which is based on the conductivity detected by the conductivity probe.

All in all, it is therefore possible to achieve (almost) continuous conveying characteristics of the metering pump using simple means. This reduces the influence of irregular conveying characteristics on the control loop of the metering pump. In particular, the use of additional mixing chambers can be avoided, which are used in known metering pumps to compensate for the disruptive influences caused by discontinuous conveying characteristics of known metering pumps.

By using the stepper motor as the electric drive of the metering pump, there is no need for a rotary encoder or position encoder for the electric drive. With a stepper motor, the position of the stepper motor can be detected without additional sensors and the stepper motor can therefore be easily and cost-effectively synchronized with the outlet and/or intake phase. This ensures a more constant fluid delivery.

Further advantageous further embodiments are described in detail below.

It is preferred if the magnetic sensor is a reed sensor or a Hall sensor. In particular, the magnetic sensor may be attached to the metering pump in order to determine the position signal of the metering pump and in particular the start of the intake and/or discharge phase. If the metering pump is a rotary vane piston pump, a position of the piston of the metering pump may be detected by the magnetic sensor. This means that the position (of the piston) of the metering pump may be determined cheaply and easily.

In a rotary vane piston pump, the piston may move between two dead centers of a piston stroke. Preferably, the magnetic sensor may detect the position signal of the metering pump at the respective dead centers. That is, the magnetic sensor may report back to the control or regulation unit when the piston is at a dead center. Preferably, positioning of the piston at the dead center corresponds to the start of the intake phase and/or of the discharge phase. The magnetic sensor may therefore determine the start of the intake phase and/or of the discharge phase.

It may be advantageous if the magnetic sensor is provided as a position determination unit to synchronize the physical position with a start of the inlet/intake phase and/or of the emission/discharge phase. For this purpose, the stepper motor may preferably be synchronized with the start of the inlet/intake phase and/or of the emission/discharge phase. The start of the inlet/intake phase and/or the emission/discharge phase may be detected by the magnetic sensor on the metering pump. The stepper motor may then be synchronized based on the detected signal.

When using a stepper motor as an electric drive, synchronization of the physical position with a magnetic sensor may therefore be sufficient (in contrast to synchronization via a rotary encoder), since a stepper motor may be set to a precise position.

The current position of the stepper motor may be determined by counting the step pulses completed after the last position signal. After initial synchronization of the position, the driving unit calculates the temporal resolution of the drive pulses for the stepper motor to achieve the best possible compensation of the intake/inlet phase and of the discharge/emission phase and generates these pulses in a required time grid.

It may therefore be advantageous to recognize the physical position of the stepper motor when implementing the driving via the driving unit. This allows the physical position of the stepper motor to be synchronized with the start of the intake phase and/or of the discharge phase. When using the stepper motor as an electric drive, the physical position of the stepper motor may be calculated from the completed steps. Alternatively, the metering pump or the stepper motor of the metering pump may be designed in such a way that the start of the intake phase and/or of the discharge phase corresponds to a dead center of the metering pump. As a result, the start of the intake phase and/or of the discharge phase is always synchronized with a piston position or a position of the stepper motor, and the stepper motor may dispense with a position determination unit such as a rotary encoder.

According to an optional aspect of the disclosure, the control or regulation unit may control or regulate a rotation speed of the stepper motor during the intake phase of the metering pump such that a rotation speed of the stepper motor during the intake phase is greater than a rotation speed of the stepper motor during the discharge phase of the metering pump.

The stepper motor of the metering pump may be controlled or regulated by the control or regulation unit in such a way that an intake-stroke rotation speed and a discharge-stroke rotation speed are not equal. The metering pump is therefore not driven with a continuous rotation speed specification, but the intake-stroke rotation speed is accelerated compared to a discharge-stroke rotation speed. This means that the intake phase is shorter than the discharge phase. The additive is therefore delivered at least approximately continuously by the metering pump and the conveying characteristics of the metering pump are smoothed.

It may be advantageous if the driving unit or the control or regulation unit is provided and configured in such a way as to limit the reduction of the intake phase to be achieved by the maximum rotation speed to be achieved by the stepper motor of the metering pump or of the first metering pump and/or of the second metering pump.

In other words, it is preferred if the preceding device performs a reduction of the electrical conductivity fluctuations during discontinuous conveying via the metering pump via different drive (motor) speeds in the intake and discharge phase.

Preferably, a frequency of the current applied to the stepper motor in the discharge phase may be lower than a frequency of the current applied to the stepper motor in the intake phase. As a result, the rotation speed of the stepper motor in the discharge phase may be lower than in the intake phase.

It is advantageous if the driving unit is provided and configured to control the stepper motor in such a way as to increase the rotation speed of the metering pump at the start and at the end of the discharge phase in accordance with a delivery characteristic of the metering pump, so that approximately constant conveying may be achieved over a plurality of intake and discharge cycles. In other words, in order to achieve the preceding aspect of driving, step pulses are generated by the driving unit during the start of the discharge phase, which reduce the rotation velocity of the stepper motor sequentially from the maximum possible rotation speed in the intake phase. At the end of the discharge phase, the rotation velocity is sequentially increased again to the maximum possible rotation speed for the intake phase, so that almost continuous conveying is created in the discharge phase.

It is also advantageous if the driving unit is configured and provided to operate the stepper motor at varying rotation speed.

The first and second metering pumps are preferably each a rotary vane piston pump (DSK).

It is advantageous if the driving unit is provided and configured to calculate a temporal resolution of drive pulses for the stepper motor in order to achieve a predetermined compensation of the intake phase and/or discharge phase.

It is advantageous if the driving unit is a microcontroller, which is preferably provided in a driver for the stepper motor of a metering pump, or if the driving unit is integrated into the regulation unit. In other words, the driving may, for example, be implemented by a microcontroller in an existing driver for a stepper motor of a metering pump or may be taken over by an existing computing unit of the existing regulation unit. The step pulses generated by the driving unit are forwarded to the hardware driver of the stepper motor, which then triggers a step movement of the stepper motor.

Furthermore, the present disclosure relates to a driving method of a metering pump for producing and/or processing dialysis liquid comprising the following steps:
detecting a position signal of a piston of the metering pump by a magnetic sensor;
synchronizing a stepper motor with a start of the intake phase and/or of the discharge phase of the metering pump, in particular by the detected position signal,
computing a temporal resolution of control pulses of the stepper motor by the control or regulation unit, such that the stepper motor rotates at a higher rotation speed in the intake phase than in the discharge phase, in particular that the stepper motor rotates at the maximum rotation speed in the intake phase,
generating the calculated control impulses in a required time grid.

In other words, the control pulses of the stepper motor as an electric drive are calculated such that the stepper motor rotates faster in the intake phase than in the discharge phase and thus the intake phase is shorter than the discharge phase.

By the driving method according to the disclosure, a metering pump may be driven in a device according to one of the preceding aspects.

The control or regulation unit outputs a manipulated variable for the stepper motor of the metering pump based on the measured actual conductivity. This output manipulated variable is very susceptible to the (regular) conductivity fluctuations caused by the irregular conveying capacity of the pump. For this reason, the manipulated variable of the control or regulation unit is changed in such a way that the control pulses shorten the intake phase and lengthen the discharge phase. This may be done either by the control or regulation unit itself or by the drive unit.

It is preferred if the driving method comprises the following steps:
generating control pulses by the driving unit during the intake phase that correspond to the maximum possible rotation speed of the stepper motor,
generating control pulses by the driving unit during the start of the discharge phase, by which the rotation velocity of the stepper motor is sequentially reduced from the maximum possible rotation speed in the intake phase,
generating step pulses by the driving unit at the end of the discharge phase in order to sequentially increase the rotation velocity back to the maximum possible rotation speed for the intake phase, so that almost continuous conveying occurs in the discharge phase.

By reducing the rotation speed at the start of the discharge phase and increasing the rotation speed at the end of the discharge phase, an almost continuous conveying capacity/ almost continuous volume flow is achieved over the entire discharge phase.

It is advantageous if the reduction of the intake phase to be achieved is limited by the maximum rotation speed to be achieved by the stepper motor of the metering pump. By using the maximum rotation speed in the intake phase, the intake phase is minimized.

It is advantageous if the control pulses generated by the driving unit are forwarded to a driver, in particular a hardware driver, of the stepper motor, which then triggers a stepping movement of the stepper motor.

The object of the present disclosure is additionally solved by a computer-readable storage medium and/or a computer program, each comprising instructions which, when executed by a computer, cause the computer to perform the steps of the driving method according to the preceding aspects.

### BRIEF DESCRIPTION OF THE DRAWINGS

FIG. 1 is a representation illustrating a control loop according to the present disclosure;

FIG. 2 is a schematic representation illustrating an operating principle of the driving unit;

FIGS. 3a to 3d are a representation illustrating the operating principle of a metering pump;

FIG. 4 is a representation illustrating a volume displacement in a rotary vane piston pump;

FIG. 5a is a representation illustrating the behavior of the pump rotation at constant rotation speed;

FIG. 5b is a representation illustrating the behavior of conductivity after the injection point;

FIG. 6a is a representation illustrating the behavior of the pump rotation at a rotation speed according to the present disclosure;

FIG. 6b is a representation illustrating the behavior of the conductivity after the injection point according to the present disclosure;

FIG. 7a is a representation illustrating the behavior of the pump rotation at a rotation speed according to the present disclosure; and

FIG. 7b is a representation illustrating the behavior of the conductivity after the injection point according to the present disclosure.

### DETAILED DESCRIPTION

Configuration examples of the present disclosure are described below on the basis of the associated Figures.

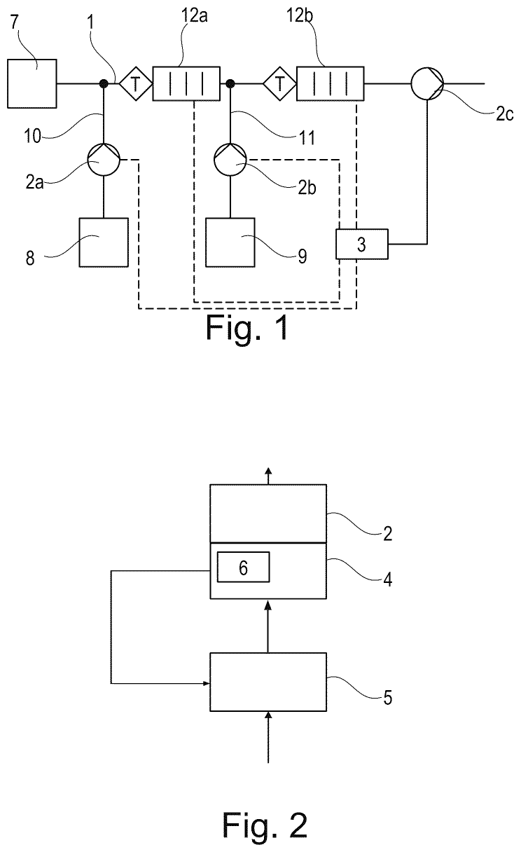

FIG. 1 is a representation illustrating a device/control loop according to the present disclosure. The device or control loop according to FIG. 1 comprises a main fluid line 1 for supplying a dialysis liquid. The main fluid line 1 starts in a first source 7, which provides the fundamental component (osmotic) water of the dialysis liquid. In a first storage means 8, the device/control loop provides a first additive or a first component, preferably a basic fluid, and in a second storage means 9, the device/control loop provides a second component/a second additive, preferably an acidic fluid.

The first storage means 8 is connected to the main fluid line 1 via a first controlled system/secondary fluid line 10. In the first controlled system 10, a first metering pump 2a is provided, which is provided and configured to convey the first component from the first storage means 8 in the direction of the main fluid line 1 and to add it thereto.

The second storage means 9 is connected to the main fluid line 1 via a second controlled system/secondary fluid line 11. In the second controlled system 11, a second metering pump 2b is provided, which is provided and configured to convey the second component from the second storage means 9 in the direction of the main fluid line 1 and to add it thereto.

The main fluid line 1 has a third metering pump 2c, which conveys the mixed dialysis fluid to a dialyzer (not shown).

In other words, the device or the control loop is provided and configured in such a way that (osmotic) water is fed from the first source 7 of the fundamental component into the main fluid line 1, which is mixed with the fluid from the first storage means 8 in a first step and mixed with the fluid from the second storage means 9 in a further step.

Both the first metering pump 2a and the second metering pump 2b are connected to a control or regulation unit 3. After adding the first component to the first storage means 8, a first probe, preferably a temperature sensor T and a conductivity probe 12a, is arranged in the main fluid line 1, which is also connected to the control or regulation unit 3. The conductivity probe 12a is provided and configured to measure the conductivity of the first (basic) component mixed with the osmotic water from the first storage means 8 after it has been added via the first controlled system 10 via the first metering pump 2a. A concentration of the first component in the liquid is determined from the conductivity detected with the aid of the temperature detected by the temperature sensor T. The control or regulation unit 3 is provided and configured to compare the actual value of the conductivity of the osmosis water with the first (basic) component with the target value and to readjust it via a rotation speed of the first metering pump 2a, if necessary.

A second probe, preferably a temperature sensor T and a conductivity probe 12b, which is also connected to the control or regulation unit 3, is arranged in the main fluid line 1 after addition of the second component from the second storage means 9. The second probe 12b is provided and configured to measure the conductivity of the second (acidic) component from the second storage means 9 after its addition via the second controlled system 11 via the second metering pump 2b in combination with the liquid already present in the main fluid line 1. In other words, the second probe 12b measures the conductivity of the entire dialysis liquid. The concentration of the second component in the dialysis liquid is preferably measured from the detected conductivity using the temperature detected by the temperature sensor T. The regulation unit 3 is provided and configured to compare the actual value of the conductivity of the entire dialysis liquid with the target value and to readjust it via a rotation speed of the second metering pump 2b, if necessary.

FIG. 2 is a schematic representation illustrating an operating principle of a driving unit 5. The driving unit 5 is either integrated into the control or regulation unit 3 or is taken over by a computing unit of the regulation unit 3 or may be implemented by a microcontroller (not shown) in an existing driver for a metering pump 2. FIG. 2 is a schematic representation or a flow chart, which applies regardless of how the driving unit 5 is implemented.

Accordingly, the metering pump 2 has a stepper motor as a drive unit/an electric drive 4, which is driven by the driving unit 5. The driving unit 5 translates the manipulated variable of the control or regulation unit 3 into an input value for the electric drive 4, for example into the control current, the angle of rotation or the number of motor steps of the electric drive 4. The metering pump 2 has a position determination unit 6, which detects a physical position of the metering pump 2.

The position determination unit 6 is a magnetic sensor. The position determination unit 6 is provided and configured to perform a synchronization of the physical position with a start of the intake phase and/or the discharge phase. The electric drive 4 is therefore synchronized with the start of the intake phase and/or of the discharge phase, which is detected by the position determination unit 6. The detected physical position of the position determination unit 6 is communicated to the driving unit 5.

After an initial synchronization of the physical position based on a conveying rate, the driving unit 5 calculates a time resolution of drive pulses for the electric drive 4 in order to achieve a predetermined compensation of the inlet/intake phase and/or discharge/emission phase. The driving unit 5 transmits step pulses to the electric drive 4 in order to increase or decrease the rotation speed of the metering pump 2 accordingly.

The driving unit 5 controls the electric drive 4 in such a way that the first metering pump 2a and/or the second metering pump 2b are provided with an accelerated rotation speed during the inlet/intake phase and a reduced rotation speed during the discharge phase or that the intake phase is shorter than the discharge phase.

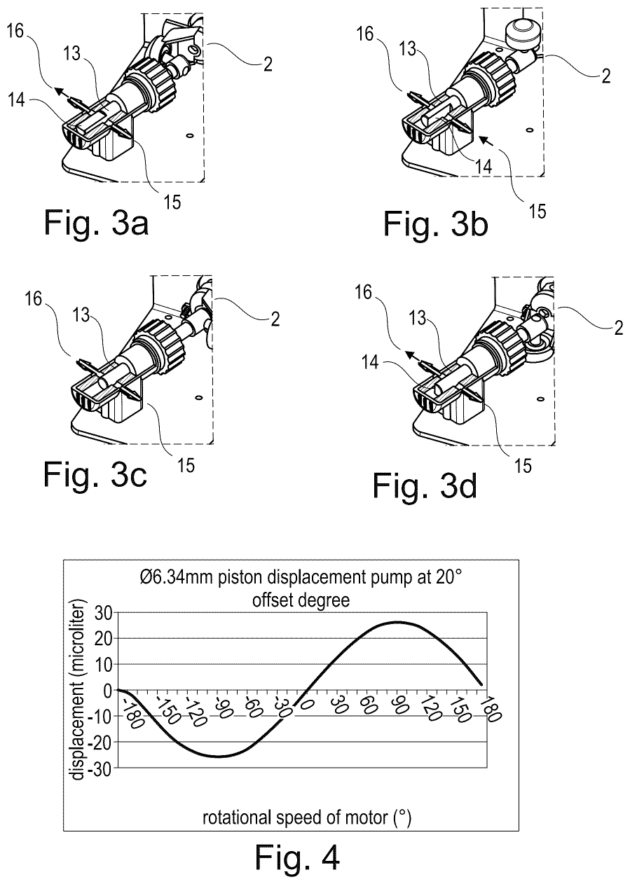

FIGS. 3a to 3d are a representation illustrating the operating principle of a metering pump 2, preferably a rotary vane piston pump, according to the present disclosure. FIGS. 3a to 3d describe four mutually different positions of a piston 13 in such a rotary vane piston pump 2. The piston 13 is cylindrical and has a flattening 14 on one side. The flattening 14 extends over at least half of the front part of the piston 13 visible in FIG. 3a.

In a first position as shown in FIG. 3a, the piston 13 is in an upper dead center position, i.e. the flattening 14 is turned/pointing upward. In the top dead center position, the metering pump 2 is provided to admit fluid/liquid.

In a second position as shown in FIG. 3b, the piston 13 retracts and thus draws fluid/liquid into the metering pump 2 via an inlet 15. In the second position, the flattening faces the inlet 15. In a third position as shown in FIG. 3c, the piston 13 is in a bottom dead center position, i.e. the flattening 14 is turned/pointing downward. In the bottom dead center position, the metering pump 2 is provided to dispense fluid/liquid via an outlet 16.

In a fourth position according to FIG. 3d, the piston 13 is in a position in which the piston 13 pushes fluid/liquid out through the outlet 16.

In other words, this means that the piston 13 and thus the flattening 14 is provided in a rotating manner, so that the rotation velocity of the piston 13 may be controlled/regulated via the rotation speed in order to be able to influence the conveying rate accordingly.

FIG. 4 is a representation illustrating a volume displacement or cylinder volume in a rotary vane piston pump 2 with a (piston) diameter of 6.34 mm at an offset angle of 20°. The x-axis shows the motor rotation speed or an angle of rotation of the electric drive 4 in degrees (°) and the y-axis shows the displacement in microliters. The curve shown corresponds to a generally known sine curve, which has a zero crossing at ±180° and 0°.

Accordingly, the sine curve shown may be divided into four portions. The first descending curve section in the range from −180° to −90° corresponds to the first position described above. The rising curve section in the range from −90° to 0° corresponds to the second position described above. The rising curve section in the range from 0° to +90° corresponds to the third position described above. The descending curve section in the range from +90° to +180° corresponds to the fourth position described above.

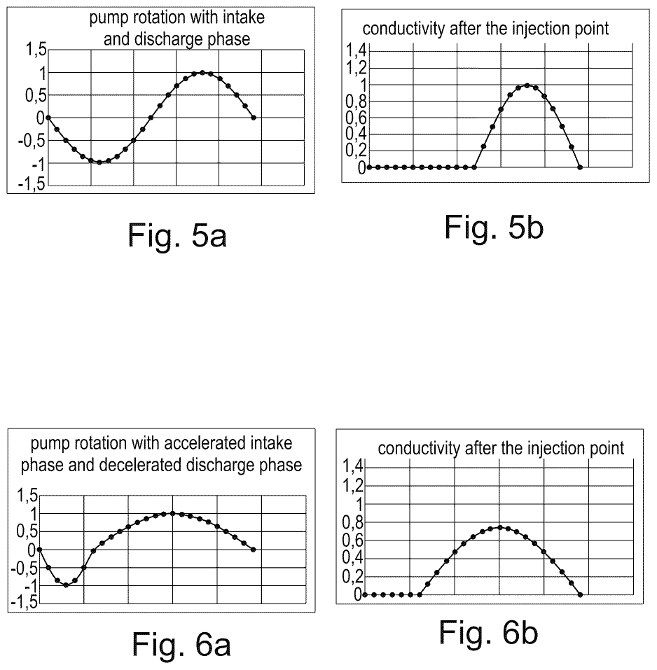

In the diagrams described below in FIGS. 5a, 5b, 6a, 6b, 7a and 7b, the pump rotation over time is shown each time and the conductivity over time is shown each time.

FIG. 5a is a representation illustrating the behavior of the pump rotation at constant rotation speed and FIG. 5b is a representation illustrating the behavior of the conductivity after the injection point as a function of the pump rotation according to FIG. 5a. Both the x-axis of FIG. 5a as well as the x-axis of FIG. 5b describe the course of time.

In FIG. 5a, a pumped volume flow of the pump over a complete pump revolution forms a generally known sinusoidal curve. The negative portion of the volume flow corresponds to the intake phase and the positive portion to the discharge/emission phase. In FIG. 5b, conductivity after the injection point is shown as a characteristic curve. Here, the conductivity is zero as long as the sinusoidal curve of the pump rotation, i.e. the pumped volume flow of the pump, is zero or negative. When the pump volume flow becomes positive, the conductivity increases and shows a parabola that opens downward. The maximum of the conductivity curve at constant rotation speed is 1.

FIG. 6a is a representation illustrating the behavior of the pump rotation at a rotation speed according to the present disclosure. FIG. 6a shows the delivered volume flow of the metering pump 2 with an accelerated rotation speed during the intake/inlet phase and a decelerated rotation speed during the discharge/emission phase. Here, the sine curve has a compressed curve in/during the inlet phase and a stretched curve in/during the discharge phase. The curve in FIG. 6b is similar. This means that the area with a conductivity not equal to zero is larger. On the other hand, the maximum conductivity over time is lower than in FIG. 5b.

FIG. 6b is a representation illustrating the behavior of the conductivity after the injection point according to the present disclosure as a function of the delivered volume flow according to FIG. 6a. In the conductivity curve of FIG. 6b as a function of the accelerated intake phase and the decelerated discharge phase, the course of the parabola opened downward is flattened in contrast to the conductivity curve at constant rotation speed. Accordingly, the maximum of the parabola opening downward of the conductivity curve is below 0.8. In this way, a more continuous conveying of fluid/liquid can be made possible.

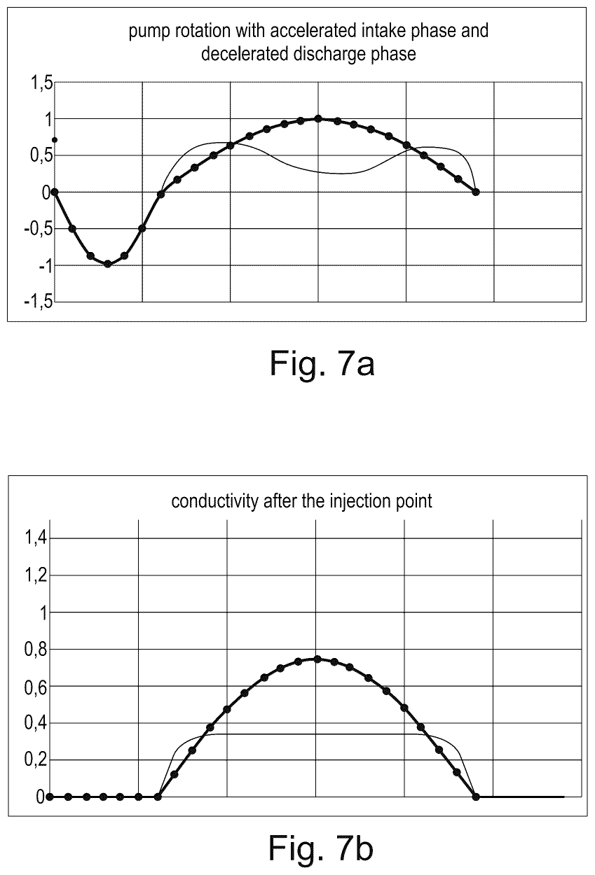

In other words, according to FIGS. 6a and 6b, the metering pump 2 is no longer driven at a continuous rotation speed, but at an accelerated rotation speed during the intake phase and a reduced rotation speed during the discharge phase. The step pulses generated by the driving unit 5 during the intake phase correspond to the maximum possible rotation speed of the drive unit 4. FIG. 7a is a representation illustrating a theoretical optimization of the pump rotation at a varying rotation speed and FIG. 7b is a representation illustrating a theoretical optimization of the conductivity according to the injection point according to the present disclosure. Both FIGS. 7a and 7b show the curve of FIGS. 6a and 6b. In comparison, a further curve is shown in each case, which is shown without individual measuring points and via a thinner line.

FIG. 7a shows a curve of the pump rotation in which the driving unit 5 generates step pulses during the start of the discharge phase, which sequentially reduce the rotation velocity of the drive unit 4 from the maximum possible rotation speed in the intake phase. At the end of the discharge phase, the rotation velocity is sequentially increased again to the maximum possible rotation speed for the intake phase, so that almost continuous conveying occurs in the discharge phase.

In other words, the curve of the pump rotation is compensated upward at the start of the discharge phase, then compensated downward again, only to be compensated upward and downward again toward the end of the discharge phase. In this way, the curve section of the discharge phase is flattened.

When looking at the conductivity curve according to FIG. 7b as a function of the pump rotation according to FIG. 7a, it can be seen that the parabola opening downward may be flattened even further by the rotation speed variations implemented above, so that a type of “plateau” may be implemented according to continuous conveying. This “plateau” has a value less than 0.4 and greater than 0.3.

### REFERENCE SIGNS

1 main fluid line
2 metering pump
3 control or regulation unit
4 electric drive (stepper motor)
5 driving unit
6 position determination unit (magnetic sensor)
7 first source
8 first storage means
9 second storage means
10 first controlled system (first secondary fluid line)
11 second controlled system (second secondary fluid line)
12 probe
13 piston
14 flattening
15 inlet
16 outlet

1.-14. (canceled)

15. A device for or of an extracorporeal blood treatment machine for producing and/or processing a dialysis liquid, the device comprising:
a main fluid line for introducing osmotic water;
at least one secondary fluid line connected to the main fluid line for supplying an additive into the osmotic water;
at least one metering pump that sucks in the additive from at least one storage container in an intake phase and discharges the additive into the osmotic water in a discharge phase that serially follows the intake phase;
an electric drive for operating the at least one metering pump; and
a control or regulation unit for controlling or regulating the electric drive in such a way that the intake phase is shorter than the discharge phase,
the electric drive comprising a stepper motor,
the at least one metering pump comprising a magnetic sensor,
the magnetic sensor operable to detect a position signal of the at least one metering pump, and
the control or regulation unit configured to synchronize a start of the intake phase and/or of the discharge phase with the position signal.

16. The device according to claim 15, wherein the position signal is of a piston of the at least one metering pump.

17. The device according to claim 16, wherein the magnetic sensor detects the position signal of the piston at respective dead centers of a piston stroke of the piston of the at least one metering pump.

18. The device according to claim 16, wherein the control or regulation unit detects a position of the piston starting from the position signal based on steps completed by the stepper motor.

19. The device according to claim 15, wherein a rotation speed of the stepper motor during the intake phase of the at least one metering pump is greater than a rotation speed of the stepper motor during the discharge phase of the at least one metering pump.

20. The device according to claim 19, wherein the control or regulation unit is configured to control the stepper motor in such a way as to increase the rotation speed of the stepper motor of the at least one metering pump at the start and at an end of the discharge phase in accordance with a delivery characteristic of the at least one metering pump.

21. The device according to claim 20, wherein the control or regulation unit is configured to control the stepper motor in such a way as to achieve constant conveying of the at least one metering pump.

22. The device according to claim 15, wherein the magnetic sensor is a reed sensor or a Hall sensor.

23. The device according to claim 15, wherein the control or regulation unit is provided and configured to calculate a temporal resolution of drive pulses for the stepper motor in order to achieve a predetermined compensation of the intake phase and/or discharge phase.

24. The device according to claim 15, wherein the control or regulation unit is a microcontroller.

25. The device according to claim 24, wherein the microcontroller is provided in a driver for the stepper motor of the at least one metering pump.

26. The device according to claim 24, wherein a driving unit is integrated into the control or regulation unit.

27. A driving method of a metering pump for producing and/or processing dialysis liquid, the driving method comprising the steps of:
detecting a position signal of a piston of the metering pump by a magnetic sensor;
synchronizing a stepper motor with a start of an intake phase and/or of a discharge phase of the metering pump;
computing a temporal resolution of control pulses of the stepper motor by a driving unit such that the stepper motor rotates at a higher rotation speed in the intake phase than in the discharge phase; and
generating the control pulses in a required time grid.

28. The driving method according to claim 27, wherein, in the synchronizing step, the stepper motor is synchronized with the position signal.

29. The driving method according to claim 27, further comprising the steps of:
generating control pulses by the driving unit during the intake phase that correspond to a maximum possible rotation speed of the stepper motor;
generating control pulses by the driving unit during the start of the discharge phase, by which a rotation velocity of the stepper motor is sequentially reduced from the maximum possible rotation speed in the intake phase; and
generating step pulses by the driving unit at an end of the discharge phase to sequentially increase the rotation velocity back to the maximum possible rotation speed for the intake phase.

30. The driving method according to claim 29, wherein the rotation velocity is sequentially increased back to the maximum possible rotation speed for the intake phase so that continuous conveying occurs in the discharge phase.

31. The driving method according to claim 29, wherein:
control pulses generated by the driving unit are forwarded to a hardware driver of the driving unit, and
the hardware driver triggers a stepping movement of the driving unit.

32. The driving method according to claim 27, wherein a reduction of the intake phase to be achieved is limited by a maximum rotation speed to be achieved by the stepper motor of the metering pump.

33. A computer-readable storage medium comprising instructions which, when executed by a computer, cause the computer to perform the driving method according to claim 27.

34. A computer program comprising instructions which, when executed by a computer, cause the computer to perform the driving method according to claim 27.
